SEQUENCE LISTING (11 sequences)

SEQ ID NO: 1 - protein, 142 aa, Homo sapiens
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 2 - DNA, 426 bp, Homo sapiens
ccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 3 - DNA, 3430 bp, Homo sapiens
ttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagccaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaagtacgccagaaagctgttgatcggcttcaagcagggaagggacacctaatttgcttttcttttctttttttttttttttttttttttttttttgagatggagttttgctcttgttgcccaggctggagtgcaatggtgcatcttggctcactacaagcctctgcctcccaggttcaagtgattctcctgcctcagcctcccaagtagctgggattacaggcacccaccaccatgcccggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcacggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctcattggactctagaatgaagccaggcagagcagggtctatgtgtgatggcacatgtggccagggtcatgcagacatgtactttgtacaaacagtgtatattgagtaaatagaaatggtgtccaggagccgaggtatcgtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgccgaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccgtctctgactttccgttctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggcagcggaactcactaaggggcccatctggacccgaggtattgtgaagataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgcgtgcatcctaagctctgagagcaaacctccctgcagggtcttgcttttaagtccaaagcctgagcccaccaaactct

SEQ ID NO: 4 - protein, 177 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 5 - DNA, 531 bp, Homo sapiens
gaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 6 - DNA, 2042 bp, Homo sapiens
tgcatcctagggacagcatagaaaggaggggcaaagtggagagagagcaacagacactgggatggtgaccccaaaacaatgagggcctagaatgacatagttgtgcttcattacggcccattcccagggctctctctcacacacacagagcccctaccagaaccagacagctctcagagcaaccctggctccaacccctcttccctttccagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcagacaagactagatccaaaaagaaaggaaccagcgcacaccatgaaggagaattgggcacctgtggttcattcttctcccagattctcagcccaacagagccaagcagctgggtcccctttctatgtggcctgtgtaactctcatctgggtggtgccccccatccccctcagtgctgccacatgccatggattgcaaggacaatgtggctgacatctgcatggcagaagaaaggaggtgctgggctgtcagaggaagctggtctgggcctgggagtctgtgccaactgcaaatctgactttacttttaattgcctatgaaaataaggtctctcatttattttcctctccctgctttctttcagactgtggctttacctcgggtaagtaagcccttccttttcctctccctctctcatggttcttgacctagaaccaaggcatgaagaactcacagacactggagggtggagggtgggagagaccagagctacctgtgcacaggtacccacctgtccttcctccgtgccaacagtgtcctaccagcaaggggtcctgtctgccaccatcctctatgagatcctgctagggaaggccaccctgtatgctgtgctggtcagcgcccttgtgttgatggccatggtaagcaggagggcaggatggggccagcaggctggaggtgacacactgacaccaagcacccagaagtatagagtccctgccaggattggagctgggcagtagggagggaagagatttcattcaggtgcctcagaagataacttgcacctctgtaggatcacagtggaagggtcatgctgggaaggagaagctggagtcaccagaaaacccaatggatgttgtgatgagccttactatttgtgtggtcaatgggccctactactttctctcaatcctcacaactcctggctcttaataacccccaaaactttctcttctgcaggtcaagagaaaggatttctgaaggcagccctggaagtggagttaggagcttctaacccgtcatggtttcaatacacattcttcttttgccagcgcttctgaagagctgctctcacctctctgcatcccaatagatatccccctatgtgcatgcacacctgcacactcacggctgaaatctccctaacccagggggaccttagcatgcctaagtgactaaaccaataaaaatgttctggtctggcctgactctgacttgtgaatgtctggatagctccttggctgtctctgaactccctgtgactctccccattcagtcaggatagaaacaagaggtattcaaggaaaatgcagactcttcacgtaagagggatgaggggcccaccttgagatcaatagcagaa

SEQ ID NO: 7 - protein, 179 aa, Homo sapiens
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 8 - protein, 178 aa, Homo sapiens
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 9 - DNA, 537 bp, Homo sapiens
gaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 10 - DNA, 534 bp, Homo sapiens
gacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 11 - DNA, 2008 bp, Homo sapiens
atggcgtagtccccaaagaacgaggacctagtaacataattgtgcttcattatggtcctttcccggccttctctctcacacatacacagagcccctaccaggaccagacagctctcagagcaaccctagccccattacctcttccctttccagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcaggtgagtggggcctggggagatgcctggaggagattaggtgagaccagctaccagggaaaatggaaagatccaggtagcggacaagactagatccagaagaaagccagagtggacaaggtgggatgatcaaggttcacagggtcagcaaagcacggtgtgcacttcccccaccaagaagcatagaggctgaatggagcacctcaagctcattcttccttcagatcctgacaccttagagctaagctttcaagtctccctgaggaccagccatacagctcagcatctgagtggtgtgcatcccattctcttctggggtcctggtttcctaagatcatagtgaccacttcgctggcactggagcagcatgagggagacagaaccagggctatcaaaggaggctgactttgtactatctgatatgcatgtgtttgtggcctgtgagtctgtgatgtaaggctcaatgtccttacaaagcagcattctctcatccatttttcttcccctgttttctttcagactgtggcttcacctccggtaagtgagtctctcctttttctctctatctttcgccgtctctgctctcgaaccagggcatggagaatccacggacacaggggcgtgagggaggccagagccacctgtgcacaggtacctacatgctctgttcttgtcaacagagtcttaccagcaaggggtcctgtctgccaccatcctctatgagatcttgctagggaaggccaccttgtatgccgtgctggtcagtgccctcgtgctgatggccatggtaaggaggagggtgggatagggcagatgatgggggcaggggatggaacatcacacatgggcataaaggaatctcagagccagagcacagcctaatatatcctatcacctcaatgaaaccataatgaagccagactggggagaaaatgcagggaatatcacagaatgcatcatgggaggatggagacaaccagcgagccctactcaaattaggcctcagagcccgcctcccctgccctactcctgctgtgccatagcccctgaaaccctgaaaatgttctctcttccacaggtcaagagaaaggattccagaggctagctccaaaaccatcccaggtcattcttcatcctcacccaggattctcctgtacctgctcccaatctgtgttcctaaaagtgattctcactctgcttctcatctcctacttacatgaatacttctctcttttttctgtttccctgaagattgagctcccaacccccaagtacgaaataggctaaaccaataaaaaattgtgtgttgggcctggttgcatttcaggagtgtctgtggagttctgctcatcactgacctatcttctgatttagggaaagcagcattcgcttggacatctgaagtgacagccctctttctctccacccaatgctgctttctcctgttcatcctgatggaagtctcaacaca

(19) United States
(12) Patent Application Publication
(10) Pub. No.: US 2018/0179490 A1
(43) Pub. Date: Jun. 28, 2018

(54) CELL COMPOSITION DEPLETED FROM TCRab and CD45RA POSITIVE CELLS

(71) Applicant: Miltenyi Biotec GmbH, Bergisch Gladback (DE)

(72) Inventors: Volker Huppert, Kürten (DE); Wing Leung, Cambridge, MA (US); Julia Dzionek, Overath (DE); Stefan Miltenyi, Bergisch Gladbach (DE)

(73) Assignee: Miltenyi Biotec GmbH, Bergisch Gladbach (DE)

(21) Appl. No.: 15/834,092

(22) Filed: Dec. 7, 2017

(30) Foreign Application Priority Data
Dec. 27, 2016 (EP) 16206909.0

(51) Int. Cl. C12N 5/00; C12N 5/0789; A61K 35/14; A61K 35/28; A61P 31/00; A61P 43/00; A61P 35/00; A61P 37/00

(52) U.S. Cl. CPC C12N 5/0087; C12N 5/0647; A61K 35/14; A61P 37/00; A61P 31/00; A61P 43/00; A61P 35/00; A61K 35/28

(57) ABSTRACT

The invention is directed to a method for the preparation of a cell population from a sample originating from bone marrow or blood, comprising the steps a) dividing the sample into a first fraction containing 50 to 99% of the cells of the sample and a second fraction containing 50 to 1% of the cells of the sample, b) labeling the cells of the first fraction with a first marker against TCR alpha/beta, c) labeling the cells of the second fraction with a second marker against CD45RA, d) removing the labeled cells from the first and second fraction and combining the remaining cells to a cell population. Furthermore, the invention is directed to a cell composition and the use of the cell composition

### FIELD OF THE INVENTION

This invention relates to a cell composition obtainable from bone marrow or blood, wherein the cell population is depleted of TCR alpha/beta positive cells and CD45RA positive cells, a method to provide such cell composition and the use thereof.

### BACKGROUND

Stem cells transplantations are more and more utilized for the treatment of hematological, oncological, immunological and genetic diseases. Usually, stem cells are extracted from bone marrow or mobilized blood processed after leukapheresis.

Depending on the donor, stem cell transplantations still suffer from complications, originating from the reaction of the transplants against the recipients (Graft-versus-host-disease, GvHD or GvHR). Other common complications include failure of engraftment of the transplanted stem cells, the toxicity of the conditioning steps, or the infections under therapy due to a prolonged or incomplete immune reconstitution.

In order to avoid complications and to tailor the stem cell transplant to a given recipient, it is known to enrich and/or deplete certain cell subpopulations from the starting cell composition. In this regard, several depletion and/or enrichment strategies are described in the prior art. Especially, the enrichment of CD34+ cells and the depletion of CD3+ cells are known processes in this field.

In a another process disclosed by US20140308250A1, a cell population obtainable from bone marrow or blood is depleted of TCR alpha/beta positive cells and simultaneously of CD19 positive cells. The thus obtained cell population may be used for the reconstitution of hematopoietic system of a human after a stem cell or bone marrow transplantation. This cell population contains additional immune cells, e.g. Natural Killer cells, gamma/delta T cells, dendritic cells, monocytes. These cell population either have an anti-tumor and/or anti-virus effect or contribute to engraftment of the stem cells. However, the cell composition obtained by the process disclosed in US20140308250A1 does not contain pathogen specific T cells, which could address infection more efficiently than NK cells and gamma/delta T cells alone.

It is known to isolate virus specific T cells from a separate cell sample based on cytokine secretion upon virus antigen encounter. This method is expensive, time consuming and results in low amounts of cells limited to reactivity against antigens used for processing.

### SUMMARY

An object of the invention was to provide a method and a cell composition that is suitable for the reconstruction of the hematopoietic system by depletion of TCR alpha/beta and CD45RA positive cells, but which still contains memory T cells, in addition to NK cells, TCRgamma/delta T cells, B cells and blood dendritic cells.

Whereas the primary application of the invention is in stem cell transplantation, the invention may also be used in other clinical settings including adoptive cellular therapy such as donor lymphocyte infusion for the treatment of infection, mixed chimerism, and cancer recurrence.

The first aspect of the present invention is a method for the preparation of a cell population from a sample originating from bone marrow or blood, comprising the steps a) dividing the sample into a first fraction containing 50 to 99% of the cells of the sample and a second fraction containing 50 to 1% of the cells of the sample, b) labeling the cells of the first fraction with a first marker against TCR alpha/beta, c) labeling the cells of the second fraction with a second marker against CD45RA, d) removing the labeled cells from the first and second fraction and combining the remaining cells to a cell population.

Another object of the invention is a pharmaceutical composition, comprising a cell population obtainable from bone marrow, whole blood or processed blood, wherein the cell population is depleted of TCR alpha/beta positive cells and CD45RA positive cells by 0.1 to 7.0 log.

Yet another object of the invention is the use or a method of use of the pharmaceutical composition for the reconstitution of the hematopoietic system of a human after a stem cell or bone marrow transplantation; or for the prevention and treatment of infection, mixed chimerism or cancer recurrence in the form of adoptive cell transfer.

### BRIEF DESCRIPTION OF THE DRAWINGS

Various exemplary details are described with reference to the following figures, wherein:

FIG. 1 shows 1 shows the IFNγ-Production by CD4+ and CD8+ T cells for the leukapheresis, TCRab depleted fraction and mixture 95:5 (TCRab depleted: CD45RA depleted) minus the negative control for each sample;

FIG. 2 shows IFNγ-Production by CD4+ and CD8+ T cells for TCRab depleted fraction and mixture 95:5 (TCRab depleted: CD45RA depleted) minus the negative control for each sample; and

FIG. 3 shows BrdU count of cells and interferon gamma content of cell culture supernatants of CD45RA negative and CD45RA positive cells upon interaction with allogeneic cells.

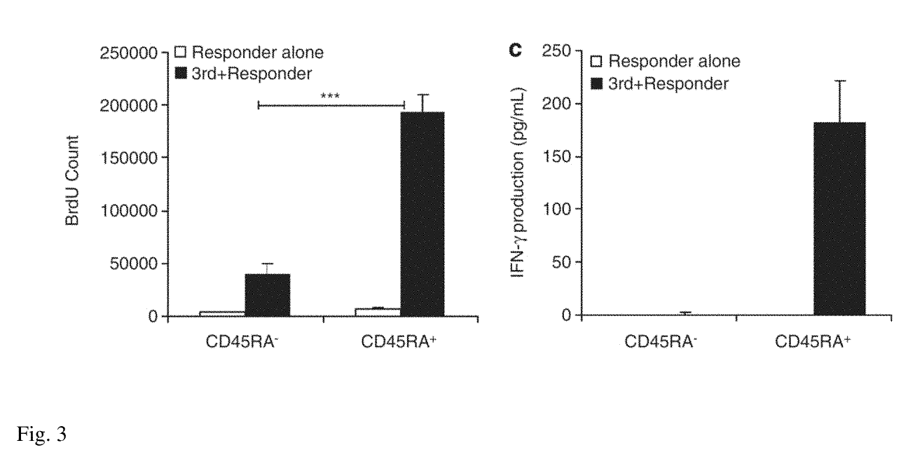

### DETAILED DESCRIPTION

The method of the invention involves the preparation of a cell population from a sample originating from bone marrow or blood. The term “sample originating from bone marrow or blood” includes all cell populations obtainable from bone marrow, fractions thereof or pre-processed bone marrow, whole blood, fractions of blood, blood products or processed blood, cell preparations obtained by leukocyte apheresis (leukapheresis), venipuncture or bone marrow puncture. Preferably, the cell preparation is obtained from a healthy donor who was previously treated with stem cell mobilizing drugs in the setting of stem cell transplantation.

The term “depletion” refers to a reduction of the amount of certain cells from a cell population. The depletion may be based on the amount of cells defined by presence (or absence) of, for example, a cell surface marker, such as TCR alpha/beta or CD45RA) by at least two logarithmic steps, preferably by at least three logarithmic steps, particularly preferred by at least 4 logarithmic steps (e.g., 4.6 logarithmic steps), most preferred by at least four to five logarithmic steps.

The removal according to logarithmic steps is as follows: 1 log:90% removal of the unwanted cells, 2 log:99%, 3 log:99.9% and 4 log:99.99%. Methods for calculating the separation performance are known to a person of skill in the art and described, for example, in Bosio et al., Isolation and Enrichment of Stem Cells, Advances in Biochemical Engineering and Biotechnology, Springer Verlag Berlin Heidelberg, 2009.

The depletion process of the invention is performed using the cell surface marker TCR alpha/beta and the surface marker CD45RA. The depletion can be performed with any technique known in state of the art, e.g. panning, elutriation or magnetic cell separation. Preferred is a depletion using magnetic cell separation, for example with the CLINIMACS Plus or CLINIMACS Prodigy instrument, both obtainable from Miltenyi Biotec GmbH due to the high depletion efficiency.

In further variants, the invention refers to a method, in particular, an in vitro method for the preparation of a population of cells. The method comprises the following steps:
providing a cell sample originating from bone marrow or blood of a donor (that is of a population that comprises, amongst others, TCR alpha/beta positive and CD45RA positive cells)
splitting the cell sample into a first and second fraction
depletion of TCR alpha/beta positive cells from the first cell population.
depletion of CD45RA positive cells from the second population
combining the TCR alpha/beta and CD45RA depleted products

Preferably, the depletion of TCR alpha/beta positive cells is performed using an antibody or antigen-binding fragment against TCR alpha/beta. On the basis of the protein or nucleotide sequences according to SEQ ID NOs 1 to 11 of the receptor TCR alpha/beta (see sequence protocols), an antibody or antigen fragment, or a derivative or conjugate thereof against TCR alpha/beta can be produced and used for the depletion of the TCR alpha/beta positive cells.

In a preferred embodiment, the method further comprises the following step: labeling cells expressing CD19 of the first fraction and/or the second fraction and/or the cell population with a third marker and removing the labeled cells.

### Labelling

The first, second and/or third marker used in the process of the invention may comprise an antibody or an antigen-binding fragment against TCR alpha/beta and/or against CD45RA and/or CD19, respectively and a detection moiety.

The detection moiety of the markers may be the same or different and may be a fluorescence dye, a magnetic particle or a radioactive label.

CD45RA is a surface molecule on naïve T cells and B cells. The term CD45RA positive cells refers to cells expressing the CD45RA molecule on the surface and to which an appropriate CD45RA -binding molecule, for example an antibody against CD45RA can specifically bind.

TCR alpha-beta is a surface molecule on T cells. The term “TCR alpha/beta positive cells” refers to a cell expressing the TCR alpha/beta molecule on the surface and to which an appropriate TCR alpha/beta-binding molecule, for example, an antibody can specifically bind.

The term “Antibody” refers to any monoclonal or polyclonal antibody of human or animal origin like be rat, rabbit, goat, horse or mice a to derivative of these antibodies that largely retains the binding capacity or the original antibody. Preferred derivatives of these antibodies are chimeric antibodies comprising, for example, chimeric antibodies of a variable region or the mouse or the rat and a human constant region. The term “antibody” comprises also bi-functional or bi-specific antibody and antibody constructs like Fvs (scFv) from single chain or antibody fusion proteins. The term “scFv” (single chain Fv Fragment) is known to a person skilled in the art and is preferred that the fragment is produced in a recombinant fashion.

Antibodies utilized in the present invention may be human or humanized. The term “humanized antibody” refers to an human antibody wherein at least one antibody binding site (complementary determining region, CDR), like for example, CDR3 and preferably all six CDRs were substituted by CDRs from a human antibody with the desired specificity. Optionally, the non-human constant region(s) was replaced by a constant region(s) of a human antibody. Methods for producing human antibodies are described for example in EP 0239400 Al and WO 90/07861 A1.

The term antigen-binding fragment refers to a fragment of an antibody as defined above like for example separated light and heavy chains, Fab, ab/c, Fv, Fab' F(ab')2. An antigen-binding fragment can comprise a variable region of the light chain and a variable region of the heavy chain, not necessarily both together.

The detection moiety of the first, second and/or third marker may be a fluorescence dye, a magnetic particle or a radioactive label. Suitable detection moieties and processes to conjugate the detection moieties to the antibody or an antigen-binding fragments are well known to the person skilled in the art.

### Depletion

In the method of the invention, the cells of the sample are divided into a first fraction and second fraction. Preferable, the first fraction contains 60% to 99%, 70% to 99% 75 to 99%, 85 to 99%, 90 to 99% or 95 to 99% of the cells of the sample, and the second fraction the respective range to add up to 100%.

After labelling the cells of the fractions as already described, the labeled cells from the first and second fraction are removed from the sample. Removal of the labeled cells is accomplished based on the nature of the detection moiety. In case the detection moiety is a fluorescence dye, removal in performed on fluorescence-based methods like FACS or using the TYTO instrument (Miltenyi Biotec GmbH). In case the detection moiety is a magnetic particle, magnetic cell sorting as known to the person skilled in the art is used. A preferred embodiment comprising magnetic cell sorting is described later.

Removal of the labeled cells may be accomplished in a first embodiment wherein in step d), first and second fraction are combined and the labeled cells are removed from the combined fraction. This strategy is preferred when the fractions contain detection moieties of a different nature like fluorescence dye and magnetic particle or if a more purer depletion is desired.

In a second embodiment of the invention, in step d), labeled cells are removed from the first and second fraction separately and the remaining cells of each fraction are combined. The second strategy is preferred when detection moieties having the same nature or even identical detection moieties are used and/or a faster processing is desired.

The process of the invention may be performed in several variants, for example:
variant A) deplete CD45RA cells from 10% cells of sample, then deplete TCRalpha/beta (or TCRalpha/beta and CD19) from 90% of sample, combine both depleted cell samples
variant B) deplete CD45RA from 10% of sample, then deplete TCRalpha/beta (or TCRalpha/beta and CD19) from 90% of sample, but retain both samples as two separate, individual products
variant C) label 10% of sample with CD45RA, then label 90% of sample with TCRalpha/beta-Biotin, combine resulting fractions, then label whole product with anti-Biotin Reagent and separate magnetically. Optionally perform wash steps between/after each labeling step
variant D) label 10% of sample with CD45RA, then label 90% of sample with TCRalpha/beta-Biotin, label with anti-Biotin Reagent, combine resulting fractions and separate magnetically. Optionally perform wash steps between/after each labeling step
variant E) magnetically depleting steps by single passage over column or two passages over column (“bulk depletion”+“sensitive depletion”) (results in highest depletion efficiency)
variant F) magnetically deplete CD45RA part by a single passage over column, magnetically deplete TCRalpha/beta part by two passages
variant G) process sample and then rebuffer into a solution feasible for infusion
variant H) process sample with infusion solution (rather than CliniMACS buffer)

In all variants of removal of labeled cells alike, the remaining cells are combined into a cell population lacking at least a part of TCR ab and CD45RA positive cells. Preferably, the total number of TCRab and CD45RA double positive cells is less than 25 thousands per kilogram recipient weight or less than 0.1% of the total nucleated cell population, like less than 0.03 or even less than 0.005%.

For example, TCRalpha/beta and CD45RA double positive T cells should be depleted by more than 99.99% (4 log) to a final cell dose of less than 25,000 TCRab+/CD45RA+ cells per kg of bodyweight of the patient, i.e. to less than 2.5 million cells for a 100 kg patient. TCRalpha/beta positive/CD45RA negative memory T cells should be retained to a safe but immunological effective dose, i.e. 0.1 million to 100 million/kg, or 10E6 to 10E9 cells for a 100 kg patient. TCRalpha/beta negative cells (both CD45RA positive and negative cell populations) should be maintained (lowest possible depletion), e.g. to a total cell dose of about 2.4E9 gamma/delta T cells (i.e. in a range or 5-250 million/kg), or 24 million gamma/delta T cells for a 100 kg patients (i.e. in a range of 500E6 to 25E9).

### Automatic Processing

In a preferred embodiment, the entire process is performed in an automated, closed system comprising storage containers for the cell sample, the labelling conjugates, washing buffer, waste and the desired cell population, one or more centrifuge chambers, detection and separation means appropriate for the detection moieties of the labelling conjugates and a tubing set for connecting purposes. The term “automated, closed system” refers to a system adapted to sterile usage i.e. air and fluid-tight closed with or without means for sterile pressure compensation and without the need of manual interaction. Such system is for example described in WO2009072003A2 or WO2009072006A2 and commercialized under the tradename CLINIMACS Plus or CLINIMACS Prodigy by Miltenyi Biotec GmbH.

Automated processing includes splitting of the starting material into a predefined split ratio for first and second fraction, determining the cell number in the sample, adding an appropriate amount of antibodies or cell separation reagents, incubating the cell product with the reagents under appropriate agitation at an appropriate temperature, removing unbound reagent by centrifugation and removal of the supernatant, removing platelet content by low speed centrifugation and removal of the supernatant, passing the cells over the magnetic separation column in a magnetic field and configure the cell product in a predefined volume and solution.

### Pharmaceutical Composition

The process of the invention can be used to obtain a pharmaceutical composition. Accordingly, another object of the invention is a pharmaceutical composition, comprising a cell population obtainable from bone marrow or blood, wherein the cell population is depleted of TCR alpha/beta positive cells and CD45RA positive cells by 0.1 log to 7.0 log.

Preferable, the pharmaceutical composition can be obtained with the method of the invention as disclosed.

The pharmaceutical composition can be used for the reconstitution of the hematopoietic system of a human after a stem cell or bone marrow transplantation; or for the prevention and treatment of infection, mixed chimerism or cancer recurrence in the form of adoptive cell transfer.

### Stem Cell and Bone Marrow Transplantation

For a bone marrow transplantation, about one liter of a bone marrow-blood mixture is removed from the pelvic bone of the donor under general anesthesia.

In order to remove stem cells from the blood, the body's own hormone-like substance is administered to the donor over several days that stimulates the production of stem cells and their transfer from the bone marrow to the blood circulatory system. The methods for the pre-treatment of the donors for the removal of bone marrow or blood stem cells are state of the art and known to the skilled artisan.

The aim of the blood stem cell transplantation is to equip the recipient with a healthy stem cell population that can differentiate into blood cells. Thereby, the deficient or the pathological cells of the recipients are being replaced. In allogeneic transplantations, the tissue stems from a healthy donor. This can be an identical sibling twin, an HLA identical sibling, a non-HLA identical family member (mismatched related donor), a haploid identical donor or an unrelated HLA-compatible donor. The main target of the allogeneic transplantation is to substitute the ill or defective hematopoietic system, like for example the bone marrow of the recipient, completely by a healthy, functional hematopoietic system comprising the immune system. The stem cell transplantation can, however, also be performed with autologous, that is, the patient's own cells.

A donor of first choice is an identical sibling (Identical Sibling:IdSib) with respect to the relevant histocompatibility antigens HLA-A, B, C, DRBI and DQB1. However, such an identical sibling can only be found in ca. 30% of the cases, such that often an HLA-identical unrelated donor (matched unrelated donor, MUD) needs to be found. Since far from all histocompatibility antigens are known and only a limited number of alleles can be tested, one needs to assume a worse match with an identical unrelated donor than with a sibling donor.

A remarkable segment of the patient population remains without donor. For these patients, related donors can be used that agree with a recipient only an one haplotype of the HLA allele, that is, haplo-identical.

Transplants of unrelated donors (MUD) are used most often for hematopoietic stem cell transplantations. For un-manipulated transplants in the MUD setting, GvHD is the main complication. Severe cases of GvHD are to be regarded as life threatening and require massive therapy with immune suppressant substances for which response rates of about 40% have been described.

### Transplantation

The actual transplantation can be divided into two phases. With the conditioning through chemo- and/or radiation therapy, the immune system of the recipient is destroyed so that the transferred or transplanted bone marrow or stem cells are not being rejected. That is to say, the recipient is being prepared for the engraftment of the transplant. The better this is achieved, the lower the risk of a non-engraftment or rejection of the transplant. Depending on the strength of the conditioning, the goal to be achieved is to destroy the remaining leukemic or malignant cells in the patient. The transplantation is performed in an intravenous manner at day 0. Until the engraftment of the transplant and the fading of the immediate toxicity, the patient remains usually in a ward suited for such a case. After the engraftment of the transplant and the waning of the immediate toxicity, a rigorous monitoring is necessary during the first three months. The intensity of the monitoring depends heavily on the type of the donor and the complications and merges into a regular life-long after care.

### Indications/Use

The cell population or pharmaceutical composition of the invention or obtained via the method of the invention may be used to treat all medical indications that require an allogeneic stem cell transplantation like inborn and acquired malignant and non-malignant diseases of hematopoietic system. The treatment may comprise allogeneic stem cell transplantation. Especially, the pharmaceutical composition according the invention or obtained with the method of the invention may be used for the reconstitution of the hematopoietic system of a human after a stem cell or bone marrow transplantation. Furthermore, the pharmaceutical composition according the invention or obtained with the method of the invention may be used for the prevention and treatment of infection, mixed chimerism, cancer recurrence, or immune disorders in the form of adoptive cell transfer with or without gene-modification or further cell manipulation. Further indications are malignant diseases that respond to a dose-intensification of the chemotherapy or radiation therapy.

During the treatment, immune suppressants like cyclosporine, corticosteroids, antimetabolites and monoclonal anti-lymphocytic antibodies may be used in order to control GvHD.

In a preferred embodiment of the pharmaceutical composition, the composition comprises further at least one pharmaceutically acceptable carrier or additive. Such carriers or additives are known to the person of skill in the art.

The pharmaceutical composition can be administered for treatment of cancer, such as, leukemia and other diseases, e.g. acute myeloid leukemia, acute lymphoblastic leukemia, aplastic anemia, thalassemia, inborn error (HHS) as well as against solid tumors (e.g. neuroblastoma, sarcoma etc.) for which an allogeneic transplantation is indicated or a therapeutic effect of TCR alpha/beta-, CD45-depleted cell preparations is to be expected.

Moreover, a sufficient amount of CD34+ cells need to be transferred (at least two, better more than four million per kg of body weight of the recipient) during an allogeneic transplantation in order to achieve a good reconstitution of the hematopoietic system. B cells can be retained in the transplant to preserve B-cell immunity if sufficient memory T cells are maintained after cell separation (for example more than 1 million CD45RA- T cells per kg). Alternatively, B cells that are removed from the transplant by CD19 depletion should be present in the smallest number possible or should be removed later in the recipient through, for example, the administration of an anti-CD20 antibody in vivo.

The amount to be administered to a human patient of the depleted cell population is typically between 2×10E10 to 1×10E11 lymphocytes.

While various details have been described in conjunction with the exemplary implementations outlined above, various alternatives, modifications, variations, improvements, and/or substantial equivalents, whether known or that are or may be presently unforeseen, may become apparent upon reviewing the foregoing disclosure. Accordingly, the exemplary implementations set forth above, are intended to be illustrative, not limiting.

### Examples

Antigen-specific T cells can support the immune defense after a stem cell transplantation and could protect the patient form different diseases (e.g. CMV, EBV, Influenza). By the expression of CD45RA and CD45R0 T cells can be divided in memory T cells, which are CD45RO+/CD45RA− and in naíve T cells which are CD45RA+/CD45RO−.

CD45RO+ memory T cells can be reactivated by repeated antigen-contact (e.g. antigens from CMV, EBV or Influenza) and produce several cytokines (e.g. IFNγ) to trigger the immune response against the infection. CD45RA depleted blood product (e.g. LP or whole blood) contains mainly CD45RO+ memory T cells, which can react against different antigens and produce INFγ.

In contrast, in TCRab-depleted cellular products, CD45RO+ memory T cells are depleted too and almost no antigen-specific T cells are expected in these cellular products.

In the method of the invention, a combination of TCRab- and CD45RA-Depletion is used as separation strategy. The target cell fraction, as an example, contains 95% of TCRab-depleted product and 5% of CD45RA depleted product.

### General Description of the Experiments

List of abbreviations: CMV: Cytomegalovirus, EBV: Ebstein-Barr-Virus, IFNγ: Interferon-gamma, LP: leukapheresis, MQ: MACSQuant Analyzer 10, SEB: Staphylococcal Enterotoxin B, TCRab: T cell receptor alpha beta

In the following experiments, a TCRab-Depletion and a CD45RA-Depletion was performed with one LP (½ LP for TCRab-Depletion and ½ LP for CD45RA). 95% of TCRab-depleted fraction and 5% of the CD45RA depleted fraction were mixed together and stimulated with different antigens. After 6 h of incubation, the INFγ production was measured by intracellular staining and measurement at the MQ. The INFγ production of the mixture 95:5 was compared to the unseparated LP and to the TCRab-depleted fraction.

### Step-By-Step Description of the Experiments

A) Split of Leukapheresis Sample
One half is used for LP-TCRab-Depletion with CliniMACS Prodigy (Miltenyi Biotec GmbH)
One half is used for CD45RA Depletion with CliniMACS Prodigy (Miltenyi Biotec GmbH)
B) After Depletion TCRab depleted fraction and CD45RA depleted fraction is mixed in a cell ratio of 95:5
C) The following fractions are used for the Rapid Cytokine Assay (detection of antigen specific T cells), performed according to the datasheet Rapid Cytokine Inspector (CD4/CD8 T Cell) Kit, 130-097-343 (Miltenyi Biotec GmbH)
   Unseparated leukapheresis
   TCRab depleted fraction
   Mixture 95:5 (TCRab depleted: CD45RA depleted fraction)
D) All fractions are stimulated with the following antigens according to the datasheet of the respective stimulation agent:
   CEF-Pool→datasheet 130-098-426 (PepTivator® CEF MHC Class I Plus) (Miltenyi Biotec GmbH)
   CMV (pp56 and IE-1 Peptivator)→datasheet 130-093-493 (Miltenyi Biotec GmbH) (PepTivator® CMV IE-1) and 130-093-438 (PepTivator® CMV pp65) (Miltenyi Biotec GmbH)
   EBV-Consensus-Pool→datasheet 130-099-764 (PepTivator® EBV Consensus) (Miltenyi Biotec GmbH)
   SEB (positive control)
   No stimulation (negative control)
E) Stimulation of cell for 6 h at 37° C., after 2 h Brefeldin A is added (Block of exocytosis)
F) Intracellular staining with anti-IFNγ-PE and Rapid Cytokine Inspector, performed according to the datasheet Rapid Cytokine Inspector (CD4/CD8 T Cell) Kit, 130-097-343 (Miltenyi Biotec GmbH)and Rapid Cytokine Inspector Anti-Cytokine Antibodies, 130-097-600 (Miltenyi Biotec GmbH)
G) Measuring of stained cells at the MACSQuant Analyzer 10 (Miltenyi Biotec GmbH)

### Results of the Experiments

The higher the frequency of IFNγ+ T cells (IFNγ-Production), the higher the reaction of antigen-specific cells and the higher the positive effect for the stem cell transplantation. This effect is expected for the mixture 95:5 but not for the TCRab-depleted target cell fraction.

The mixture 95:5 (TCRab depleted: CD45RA depleted fraction) shows a production of IFNγ after stimulation with CEF-Pool and CMV compared to the TCRab depleted fraction. This show that the mixture 95:5 contains antigen-specific T cells against CMV and peptides from CEF-Pool (CMV, EBV, Influenza), which is an advantage compared to the TCRab-depleted fraction.

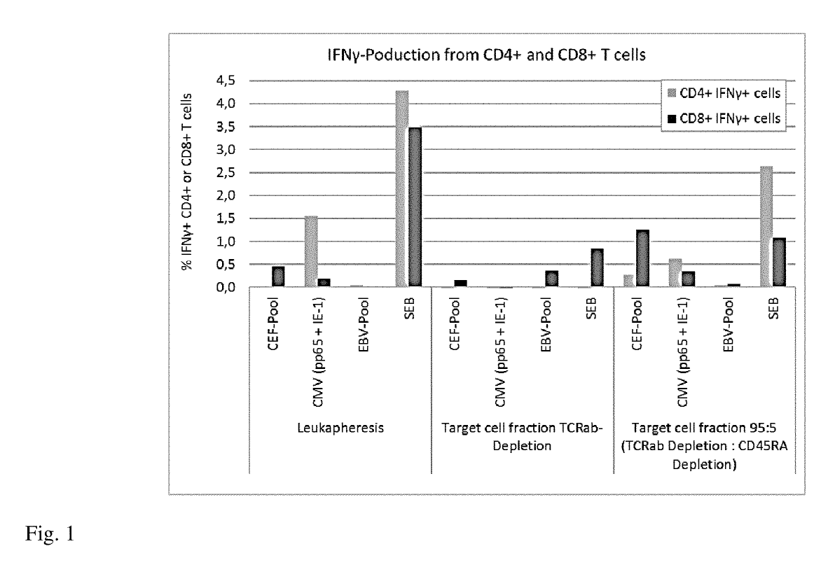

FIG. 1 shows the IFNγ-Production by CD4+ and CD8+ T cells for the leukapheresis, TCRab depleted fraction and mixture 95:5 (TCRab depleted: CD45RA depleted) minus the negative control for each sample. The positive effect of IFNγ-production can be seen from the column “mixture 95:5 (TCRab depleted: CD45RA depleted)” as compared to the TCRab depleted fraction.

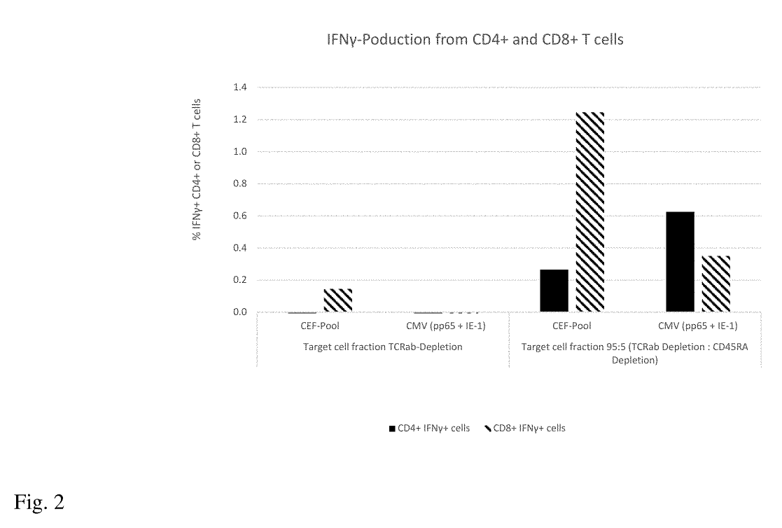

FIG. 2 shows IFNγ-Production by CD4+ and CD8+ T cells for TCRab depleted fraction and mixture 95:5 (TCRab depleted: CD45RA depleted) minus the negative control for each sample. The cells from the TCRab depleted fraction shown no reaction, i.e. no IFNγ-Production, whereas cells obtained by the method of the invention show the positive effect of IFNγ-Production.

The antigen-specific T cells obtained with the method of the invention can support the immune defense after a stem cell transplantation and could protect the patient form different diseases (e.g. CMV, EBV, Influenza).

### Reduction of Alloreactivity

Non-processed apheresis products contain alloreactive T cells, possibly resulting in severe side effects after infusion to a patient. Alloreactivity is removed by TCRalpha/beta depletion. The combination of TCRalpha/beta depleted and CD45RA depleted products could contain alloreactive cells if they are included in the CD45RA depleted product.

Procedure: Peripheral blood mononuclear cells were separated into CD45RA positive and CD45RA negative cells by magnetic cell sorting. Gamma irradiated third party PBMCs were used as stimulator cells to activate CD45RA positive or negative responder cells in a co-culture system. Proliferative response of CD45RA positive and negative cells was assessed by BrdU count. Activation state was assessed by quantification of Interferon-gamma in the cell culture supernatants. Three independent experiments were performed.

Results: CD45RA positive cells had a significantly higher BrdU count compared to CD45RA negative cells (200000 vs. 40000), indicating for active proliferation in CD45RA positive but not CD45RA negative cell upon interaction with allogeneic cells. Interferon gamma was detected in the supernatant of CD45RA positive but not CD45RA negative cells upon interaction with allogeneic cells.

Conclusion: Addition of CD45RA depleted cells to a TCRalpha/beta depleted stem cell product does not add unwanted alloreactive potential to the stem cell product.

### Manufacturing of Cell Product/Pharmaceutical Composition

On CliniMACS plus: Set separation program DEPLETION 3.1 and use CliniMACS Depletion Tubing Set. CD45RA labelled cells (5%) were added to the re-application bag shortly before sensitive depletion of a DEPLETION 3.1/Depletion Tubing Set run with a TCRab labelled product was completed.

Composition Before Depletion:
24.5% TCRab+/CD45RA+ cells (of 1.12E10=2.74E9)

Composition After depletion:
0.03% TCRab+/CD45RA+ cells (of 3.33E9=1E6), 3.43 log depletion of TCRab+/CD45RA+ cells

While various details have been described in conjunction with the exemplary implementations outlined above, various alternatives, modifications, variations, improvements, and/or substantial equivalents, whether known or that are or may be presently unforeseen, may become apparent upon reviewing the foregoing disclosure. Accordingly, the exemplary implementations set forth above, are intended to be illustrative, not limiting.

What is claimed is:

1. A method for the preparation of a cell population from a sample originating from bone marrow or blood, comprising the steps a) dividing the sample into a first fraction containing 50 to 99% of the cells of the sample and a second fraction containing 50 to 1% of the cells of the sample, b) labeling the cells of the first fraction with a first marker against TCR alpha/beta, c) labeling the cells of the second fraction with a second marker against CD45RA, d) removing the labeled cells from the first and second fraction and combining the remaining cells to a cell population.

2. The method according to claim 1, wherein the first marker comprises an antibody or antigen-binding fragment against TCR alpha/beta and a detection moiety.

3. The method according to claim 1, wherein the second marker comprises an antibody or antigen-binding fragment against CD45RA and a detection moiety.

4. The method according to claim 1, characterized in labeling cells of the first fraction and/or the second fraction and/or the cell population with a third marker against CD19 and removing the labeled cells.

5. The method according to claim 4, wherein the third marker comprises an antibody or antigen-binding fragment against CD19 and a detection moiety.

6. The method of the claim 1, wherein the detection moiety of the first and/or second and/or third marker is a fluorescence dye, a magnetic particle or a radioactive label.

7. The method of the claim 1, wherein in step d), first and second fraction are combined and the labeled cells are removed from the combined fraction.

8. The method of claim 1, wherein in step d), labeled cells are removed from the first and second fraction separately and the remaining cells of each fraction are combined.

9. A pharmaceutical composition, comprising a cell population obtainable from bone marrow, whole blood or processed blood, wherein the cell population is depleted of TCR alpha/beta positive cells and CD45RA positive cells by 0.1 to 7.0 log.

10. The pharmaceutical composition according to claim 9, wherein the cell population is obtained with the method according to claim 1.

11. A use of the pharmaceutical composition according to claim 9 for the reconstitution of the hematopoietic system of a human after a stem cell or bone marrow transplantation.

12. A use of the pharmaceutical composition according to claim 9 for the prevention and treatment of infection, mixed chimerism, cancer recurrence, or immune disorders.
